(19) United States
(12) Patent Application Publication
(10) Pub. No.: US 2016/0015818 A1
(43) Pub. Date: Jan. 21, 2016

(54) COMPOSITIONS AND METHODS FOR PHYSIOLOGICAL DELIVERY USING CANNABIDIOL

(71) Applicant: Medipath, Inc., Newport Beach, CA (US)

(72) Inventor: Ashraf Taha, Irvine, CA (US)

(73) Assignee: Medipath, Inc., Newport Beach, CA (US)

(21) Appl. No.: 14/804,125

(22) Filed: Jul. 20, 2015

Related U.S. Application Data
(60) Provisional application No. 62/026,451, filed on Jul. 18, 2014.

(51) Int. Cl. A61K 47/46; A61K 9/00; A61K 31/4458; A61K 31/195; A61K 31/135; A61K 31/4168; A61K 31/192; A61K 31/196; A61K 31/5415; A61K 31/407; A61K 31/4422; A61K 31/277; A61K 31/137; A61K 31/55; A61K 31/60; A61K 31/327; A61K 31/167

(52) U.S. Cl. CPC A61K 47/46; A61K 31/167; A61K 9/0014; A61K 31/4458; A61K 31/195; A61K 31/135; A61K 31/4168; A61K 31/192; A61K 31/196; A61K 31/5415; A61K 31/407; A61K 31/4422; A61K 31/277; A61K 31/137; A61K 31/55; A61K 31/60; A61K 31/327; A61K 9/0053

(57) ABSTRACT

The present invention relates to compositions and methods for the administration of Cannabinoids to a patient, and in a specific embodiment, the compositions and methods may utilize or include cannabinoids, and one or more active pharmaceutical ingredients, wherein said composition is configured for transdermal or oral delivery.

### CROSS REFERENCE TO RELATED APPLICATIONS

This application claims the benefit of and priority to U.S. Provisional Application Ser. No. 62/026,451, filed Jul. 18, 2014, the contents of which are incorporated herein by reference.

### TECHNICAL FIELD AND BACKGROUND

Many medical products and associated methods have used traditional means of drug delivery, including by way of example, oral delivery, intravenous injections, subcutaneous injections, and/or intramuscular injection. Another means of drug delivery is transdermal drug technology. It has a unique advantage to oral medications because of its first pass ability to bypass the liver for breakdown meaning that the patient requires less drugs as well as have an immediate local event and effect as opposed to a systemic approach from oral medications.

Relatedly, the cannabis plant which is highly lipophilic, contains three different species, Cannabis sativa, Cannabis indica and Cannabis ruderalis. The present disclosure combines Cannabidiol (CBD) and other isolated cannabinoids like, for example, Cannabinol (CBN) and non-Tetrahydrocannabinol (THC) or very low THC parts of the Cannabis plant species utilizing their lipophilic properties used in a topical combination with Active Pharmaceutical ingredients (APIs) providing an improved multipurpose transdermal compound for medicinal value.

### BRIEF DESCRIPTION

By way of example and not limitation, one aspect of a composition is disclosed. A composition includes cannabinoids, and one or more active pharmaceutical ingredients, wherein said composition is configured for transdermal delivery.

One aspect of a method for facilitating the transdermal delivery of cannabinoids is also disclosed. The method includes providing transdermal delivery of cannabidiol to a patient in need thereof, wherein said method comprises administering a composition according to claim 1 to the patient.

Yet another aspect of a composition is also disclosed. A composition includes cannabinoids, and one or more active pharmaceutical ingredients, wherein said composition is configured for oral delivery.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates a cross section of a membrane that is made of several layers.

FIG. 2 illustrates a cross section of a membrane depicting the presence of a combination of cannabinoids and APIs as a topical cream or ointment.

FIG. 3 illustrates a cross section of a membrane that exemplifies the lipophilic properties of an ointment or cream as it enters and is absorbed by the lower layers of the membrane.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase in one embodiment is utilized repeatedly. The phrase generally does not refer to the same embodiment, however, it may. The terms comprising, having and including are synonymous, unless the context dictates otherwise.

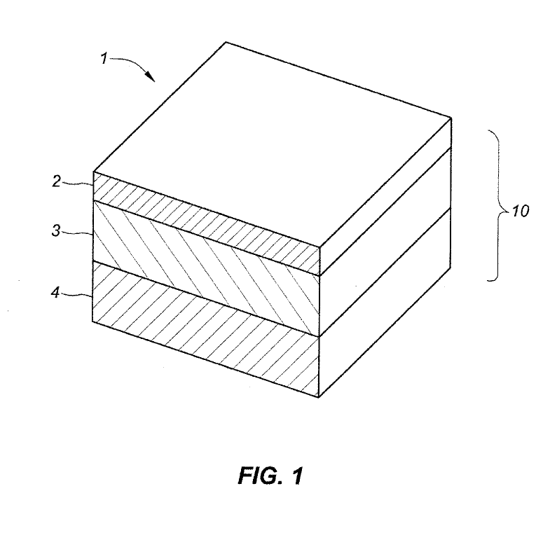

Representative FIG. 1 illustrates a cross section of a membrane 10 that is made of several layers. As discussed herein, the membrane may be the skin of any physiological being, including human or animal. The three layers of skin are labeled herein as epidermis 2, dermis 3, and hypodermis 4. The epidermis 2 is composed of multiple layers. The outermost portion of the epidermis is the stratum corneum 1 and is made of dead cells.

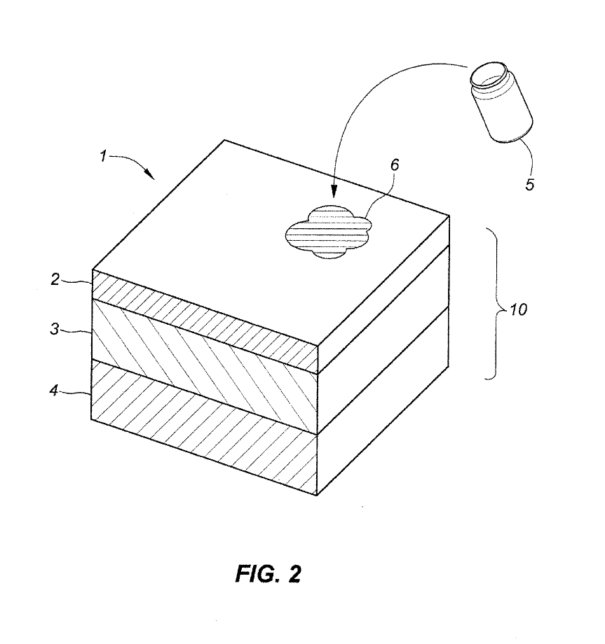

Representative FIG. 2 illustrates a cross section of a membrane depicting the presence of a combination of cannabinoids and APIs as a topical cream or ointment. Similar as with respect to FIG. 1, above, the three layers of skin are labeled epidermis 2, dermis 3, and hypodermis 4. The epidermis 2 is composed of multiple layers. The outermost portion of the epidermis is the stratum corneum 1 and is made of dead cells. The combined cannabinoid and API cream bottle 5 contains the combined cannabinoid and API lipophilic cream that is placed topically on the stratum corneum 1.

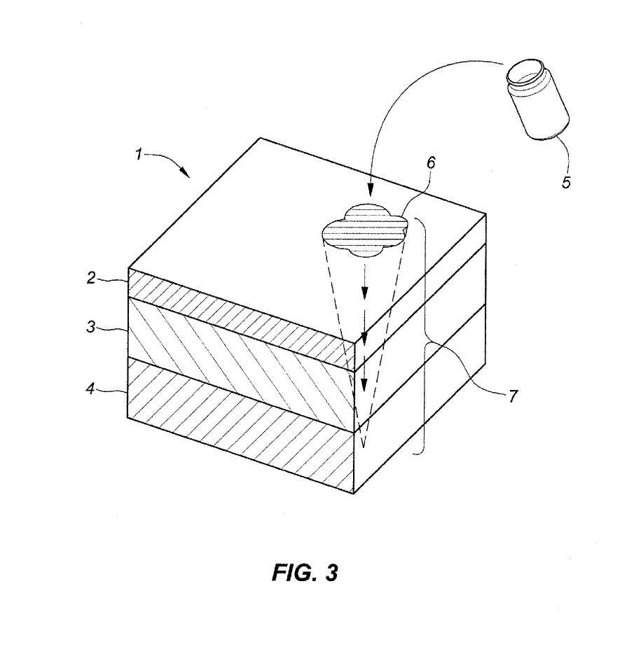

Representative FIG. 3 illustrates a cross section of a membrane that exemplifies the lipophilic properties of an ointment or cream as it enters and is absorbed by the lower layers of the membrane. The dotted lines and arrows 7 represent the lipophilic ability of the cannabinoid to absorb into the lower layers of the epidermis 2, dermis 3, and hypodermis 4 carrying the APIs to desired target area.

As used consistently throughout this disclosure, Cannabinoids will be used herein to refer to Cannabidol (CBD) and other isolated cannabinoids like Cannabinol (CBN) and non-Tetrahydrocannabinol (THC), or very low THC, parts of the Cannabis plant species including by way of non-limiting example Cannabis sativa (including hemp), Cannabis indica and Cannabis ruderalis and all resins, stalks, flowers, seeds and oils related thereto.

Likewise, Active Pharmaceutical Ingredients (APIs) may refer to pharmaceuticals from natural origin such as plant or herbal or mineral origin, chemical drug from natural origin, drug derived from chemical synthesis, drug derived from animal origin such as hormones, drug derived from microbial origin such as antibiotics, drug derived from biotechnology genetic engineering, and drugs derived from radioactive substances.

As a person of ordinary skill in the art may appreciate, Cannabinoids are known to be extremely lipophilic. Cannabinoids when used as a transdermal pharmaceutical drug transporter for Active Pharmaceutical Ingredients (API) may enable the body to receive medications that can cross transdermally and directly into the blood stream. The use of a natural product such as Cannabinoids as a lipophilic agent for APIs may reduce the side effects of synthetic creams used today for similar purposes as well as provide its own independent medical benefit associated with CBD.

The use of transdermal creams for pharmaceutical delivery of APIs has had many years of published reports and successful use. Transdermal creams target peripheral local systems while systemic absorption remains low giving a more targeted approach to treating the symptoms and pathology. Common uses for transdermal technology may include Rheumatoid Arthritis, Joint pain, inflammation, plantar fasciitis, migraines, muscle cramps, muscle pain, colitis, Irritable Bowel Syndrome (IBS), PTSD, Fibromyalgia, Radiation Proctitis, Diaper rash, Neuropathic Pain, neuropathy in general, Opiod Tolerance, Constipation with Opiods, wound care, Radiation burns, others burns, Amputation pain and Inflammatory Pain from injury.

Many formulations can be created with one or more APIs mixed with Cannabinoid(s) lipophilic transdermal system. These combinations may include amitriptyline HCL 2%, Baclofen 5%, Ketoprofen 10%, Lidocaine 2%, Lidocaine 5%, Lidocaine 10%, Clonidine 0.2%, Piroxicam 5%, Piroxicam 2%, Diclofenac Sodium 10%, Guasifenesisin 2%, Ketamine HCL 5%, Allantion 2%, and Bupivacaine HCL 1%. Many other topical APIs may also be used without departing from the teachings disclosed herein. By way of further example, the following chart sets forth common topical compounding APIs that may be used with Cannabinoids used as a lipophilic transporter across the dermis.

| API → Percent | | Pathology and symptom |
|---|---|---|
| Ranges, Drug and | Main Mechanism | treatments (not inclusive |
| Dose | of Action | of all) |
| Lidocaine 1-15% | Sodium channel | Neuropathic, Arthritic, |
| | Blocker | Acute pain, and |
| | | Inflammatory Pain |
| Buvicaine HCL 1%-3% | Sodium Channel | Neuropathic, Arthritic, |
| | Blocker | Acute pain, and |
| | | Inflammatory Pain |
| Gabapentin 5-15% | Sodium channel | Neuropathic and |
| | Blocker and | Inflammatory Pain |
| | Glutamate channel | |
| | Blocker | |
| Ketamine 5-15% | NMDA-Ca Channel | Radicular pain; cervical and |
| | Blocker | lumbar, allodynia, |
| | | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, phantom pain |
| | | syndrome, post-herpatic |
| | | pain Neuralgia. |
| Cloinidine 0.2-1% | Alpha-2 Agonist | Neuropathic pain, Chronic |
| | | pain, Trigeminal Neuralgia, |
| | | peripheral neuropathy, |
| | | diabetic ulcer pain, Post-OP |
| | | Neuropathic pain, complex |
| | | Regional Pain syndrome, |
| | | phantom pain syndrome, |
| | | post herpatic pain |
| | | Neuralgia |
| Ketoprofen 5-20%% | Non-Steriodal Anti- | Radicular pain; cervical and |
| | Inflammatory Drugs | lumbar, allodynia, |
| | (NSAIDs) | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, Muscular pain, |
| | | osteoarthritis, Joint pain, |
| | | Rheumatoid arthritis, |
| | | Fibromyalgiahantom pain |
| | | syndrome, post-herpatic |
| | | pain Neuralgia |
| Dicofenac 2-20%% | Non-Steriodal Anti- | Radicular pain; cervical and |
| | Inflammatory Drugs | lumbar, allodynia, |
| | (NSAIDs) | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, Muscular pain, |
| | | osteoarthritis, Joint pain, |
| | | Rheumatoid arthritis, |
| | | Fibromyalgiahantom pain |
| | | syndrome, post-herpatic |
| | | pain Neuralgia |
| Piroxicam 5%-10% | Non-Steriodal Anti- | Radicular pain; cervical and |
| | Inflammatory Drugs | lumbar, allodynia, |
| | (NSAIDs) | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, Muscular pain, |
| | | osteoarthritis, Joint pain, |
| | | Rheumatoid arthritis, |
| | | Fibromyalgiahantom pain |
| | | syndrome, post-herpatic |
| | | pain Neuralgia |
| Ketorolac .5%-3% | Non-Steriodal Anti- | Radicular pain; cervical and |
| | Inflammatory Drugs | lumbar, allodynia, |
| | (NSAIDs | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, Muscular pain, |
| | | osteoarthritis, Joint pain, |
| | | Rheumatoid arthritis, |
| | | Fibromyalgiahantom pain |
| | | syndrome, post-herpatic |
| | | pain Neuralgia |
| Nifedipine 2-20% | Calcium Channel | Diabetic Neuropathy, |
| | Blocker | Peripheral blood flow and |
| | | circulation, |
| Verapamil 5-15% | Calcium channel | Fibrosis/Scarring |
| | Blocker | |
| Amitriptyline 2-15% | Tricyclic | Radicular pain; cervical and |
| | Antidepressant | lumbar, allodynia, |
| | | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, Muscular pain, |
| | | osteoarthritis, Joint pain, |
| | | Rheumatoid arthritis, |
| | | Fibromyalgiahantom pain |
| | | syndrome, post-herpatic |
| | | pain Neuralgia |
| Imipramine 2-15% | Tricyclic | Radicular pain; cervical and |
| | Antidepressant | lumbar, allodynia, |
| | | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, Muscular pain, |
| | | osteoarthritis, Joint pain, |
| | | Rheumatoid arthritis, |
| | | Fibromyalgiahantom pain |
| | | syndrome, post-herpatic |
| | | pain Neuralgia |
| Cyclobenzaprine 2-5% | Tricyclic | Radicular pain; cervical and |
| | Antidepressant | lumbar, allodynia, |
| | | Hyperalgesia, Neuropathic |
| | | pain, Chronic pain, |
| | | peripheral neuropathy, |
| | | diabetic ulcer pain, Post |
| | | OP Neuropathic pain, |
| | | complex Regional Pain |
| | | syndrome, Muscular pain, |
| | | osteoarthritis, Joint pain, |
| | | Rheumatoid arthritis, |
| | | Fibromyalgiahantom pain |
| | | syndrome, post-herpatic |
| | | pain Neuralgia, muscle |
| | | relaxant |
| Baclofen 2-10% | Gaba-B Agonist | Muscle relaxant, |
| | | Fibromyalgia, TMJ pain |
| Salicylic Acid 0.5-10% | Beta hydroxyl acid | Acne |
| Benzoyl Peroxide | | Acne |
| 0.5%-10% | | |

Herein and throughout, pharmaceutical agents can refer to drugs from natural origin such as plan or herbal or mineral origin, chemical drug from natural origin, drug derived from chemical synthesis, drug derived from animal origin such as hormones, drug derived from microbial origin such as antibiotics, drug derived from biotechnology genetic engineering and drugs derived from radioactive substances. Although, as herein described, transdermal transport of CBD is a preferred embodiment, alternative preferred embodiments may be readily apparent to a person of ordinary skill, including delivering CBD orally to a patient in pill or capsule form.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A composition consisting of:
cannabinoids; and
one or more active pharmaceutical ingredients;
wherein said composition is configured for transdermal delivery.

2. The composition of claim 1, wherein the transdermal delivery form is a cream.

3. The composition of claim 1, wherein the transdermal delivery is an ointment.

4. The composition of claim 1, wherein the transdermal delivery is in the form of a patch.

5. The composition of claim 1, wherein the source of cannabinoids is one or more selected from the group consisting of Cannabis sativa, Cannabis indica and Cannabis ruderalis.

6. The composition of claim 1, wherein the one or more active pharmaceutical ingredients include one or more selected from the group consisting of Amitriptyline HCL 2-15%, Baclofen 2-10%, Ketoprofen 5-20%, Lidocaine 1-15%, Clonidine 0.2-1%, Gabapentin 5-15%, Piroxicam 5%-10%, Diclofenac Sodium 2-10%, Guasifenesisin 2%, Ketamine HCL 5-15%, Allantion 2%, Bupivacaine HCL 1-3%, Ketorolac 0.5%-3%, Nifedipine 2-20%, Verapamil 5-15%, Imipramine 2-15%, Cyclobenzaprine 2-5%, Salicylic Acid 0.5-10% and Benzoyl Peroxide 0.5%-10%

7. A method comprising:
providing transdermal delivery of cannabinoids to a patient in need thereof, wherein said method comprises administering a composition according to claim 1 to the patient.

8. The method of claim 7, wherein the patient is a human, and the composition is administered to the human to address one or more of Rheumatoid Arthritis, joint pain, inflammation, plantar fasciitis, migraines, muscle cramps, muscle pain, colitis, Irritable Bowel Syndrome(IBS), Post Traumatic Stress Disorder (PTSD), fibromyalgia, radiation proctitis, diaper rash, neuropathic pain, neuropathy in general, opiod tolerance, phantom pain, herpatic pain, constipation with opiods, wound care, radiation burns, amputation pain and inflammatory pain.

9. The method of claim 8, wherein the composition is administered once daily.

10. The method of claim 8, wherein the composition comprises different strengths and strains.

11. A composition consisting of:
cannabinoids; and
one or more active pharmaceutical ingredients;
wherein said composition is configured for oral delivery.

12. The composition of claim 11, wherein the oral delivery form is a capsule.

13. The composition of claim 11, wherein the oral delivery form is an pill.

14. The composition of claim 11, wherein the source of cannabinoids is one or more selected from the group consisting of Cannabis sativa, Cannabis indica and Cannabis ruderalis.

15. The composition of claim 11, wherein the one or more active pharmaceutical ingredients include one or more selected from the group consisting of Amitriptyline HCL 2-15%, Baclofen 2-10%, Ketoprofen 5-20%, Lidocaine 1-15%, Clonidine 0.2-1%, Gabapentin 5-15%, Piroxicam 5%-10%, Diclofenac Sodium 2-10%, Guasifenesisin 2%, Ketamine HCL 5-15%, Allantion 2%, Bupivacaine HCL 1-3%, Ketorolac 0.5%-3%, Nifedipine 2-20%, Verapamil 5-15%, Imipramine 2-15%, and Cyclobenzaprine 2-5%, Salicylic Acid 0.5-10%, and Benzoyl Peroxide 0.5%-10%.
